(19) United States
(12) Patent Application Publication
(10) Pub. No.: US 2025/0144059 A1
(43) Pub. Date: May 8, 2025

(54) GUANIDINOACETIC ACID, ITS DERIVATES, COMPOSITIONS AND METHODS OF USE THEREOF

(71) Applicants: Ruey J. YU, Chalfont, PA (US); Eugene J. VAN SCOTT, Abington, PA (US); Kenneth YU, Orlando, FL (US); Christopher STAHLEY, Orlando, FL (US)

(72) Inventors: Ruey J. YU, Chalfont, PA (US); Eugene J. VAN SCOTT, Abington, PA (US); Kenneth YU, Orlando, FL (US); Christopher STAHLEY, Orlando, FL (US)

(21) Appl. No.: 18/938,440

(22) Filed: Nov. 6, 2024

Related U.S. Application Data
(60) Provisional application No. 63/596,717, filed on Nov. 7, 2023.

(51) Int. Cl. A61K 31/197; A61K 9/06; A61K 45/06; A61K 47/10; A61K 47/12; A61K 47/44; A61P 17/04; A61P 25/02

(52) U.S. Cl. CPC A61K 31/197; A61K 9/06; A61K 45/06; A61K 47/10; A61K 47/12; A61K 47/44; A61P 17/04; A61P 25/02

(57) ABSTRACT

Provided herein are guanidinoacetic acid or its derivatives thereof, compositions comprising the same, as well as the use thereof, particularly for topical or systematic treatment of pain, itch, eczema or other inflammatory disorders.

### CROSS-REFERENCE TO RELATED APPLICATIONS

This application claims priority to U.S. Patent Application No. 63/596,717, filed on Nov. 7, 2023, the disclosure of which is incorporated herein by reference in its entirety.

### TECHNICAL FIELD

The present disclosure relates to compounds of guanidinoacetic acid and its derivatives, compositions comprising the same, and uses thereof for topical or systemic administration to treat pain, itch, or eczema, or inflammation associated with a cosmetic or a medical condition, disorder or disease in a human subject. In addition, the present disclosure describes clinical use of these compounds and compositions which can reduce or eliminate erythema, edema and tissue distortions associated with inflammation.

### BACKGROUND

Based on the Merck Index 15^{th} Edition 2013, item #4530, guanidinoacetic acid, also known as glycocyamine, is a white crystalline compound, which is fairly soluble in water with a molecular weight of 117. Guanidinoacetic acid is one of the precursor compounds of creatine. Guanidinoacetic acid can be converted to creatine in the liver by N-methyltransferase enzyme and has the following chemical formula (1):

H₂NC(═NH)NHCH₂COOH FORMULA (1)

It is noted that guanidinoacetic acid lacks one methyl group from creatine.

### BRIEF SUMMARY OF THE PRESENT DISCLOSURE

The inventors of the present disclosure have discovered that guanidinoacetic acid or its derivative, with or without other pharmacological agents, is cosmetically or therapeutically effective for alleviating or eliminating pain, itch, eczema or discomfort associated with an inflammatory cosmetic or a medical condition, disorder, or disease when administered topically or systemically to a human subject.

More specifically, the inventors discovered that guanidinoacetic acid or its derivative, is as therapeutically effective as analgesic substances for eradicating pain, itch, and eczema associated with inflammatory neuropathic pain, itch, eczema, arthritis, migraine headache, acute common headache, osteoarthritis, psoriatic arthritis, rheumatoid arthritis and various other pain, itch, and or eczemas associated with inflammation in human subjects, when topically or systemically administered. The inventors have discovered and repeatedly confirmed that guanidinoacetic acid or its derivative, is therapeutically effective for superficially subcutaneous injection or systemic administration to alleviate or eradicate pain, itch, eczema associated with an inflammatory medical condition, disorder or disease in human subjects. The systemic administration includes parenteral injections, oral or nasal spray, under the tongue administration (i.e., sublingual) to bypass liver digestion with oral administration.

In one general aspect, the present disclosure relates to a guanidinoacetic acid or its derivative of Formula (2),

H₂NC(═NR₁)NHCH₂COR₂ FORMULA (2),

or a composition comprising the same, wherein,
R₁ is an acyl radical having up to 29 carbon atoms;
R₂ is OR₃, NHR₄, or any amino group containing a radical having up to 29 carbon atoms;
R₃ is H, an alkyl, aralkyl or aryl radical, wherein the radical has up to 19 carbon atoms; and
R₄is H, OH, an alkyl, aralkyl, aryl or acyl radical, wherein the radical has up to 19 carbon atoms.

A typical acyl radical and abbreviation suitable for use in the present disclosure includes, but is not limited to, Ac (acetyl), isoBa(isobutanoyl), Pa(propanoyl), Bz(benzoyl), Pc(phenylacetyl), Ab(2-acetoxybenzoyl) and Pg(pyroGlutamyl).

In one embodiment, the guanidinoacetic acid or its derivative of Formula (2) is selected from the following compounds:

| Number | Name | Formula |
|---|---|---|
| G10 | guanidinoacetic acid | H₂NC(═NH)NHCH₂COOH |
| G11 | N-acetyl ethyl guanidinoacetate | H₂NC(═NAc)NHCH₂COOC₂H₅ |
| G12 | N-propanoyl ethyl guanidinoacetate | H₂NC(═NPa)NHCH₂COOC₂H₅ |
| G13 | N-propanoyl methyl guanidinoacetate | H₂NC(═NPa)NHCH₂COOCH₃ |
| G14 | N-benzoyl ethyl guanidinoacetate | H₂NC(═NBz)NHCH₂COOC₂H₅ |
| G15 | N-benzoyl methyl guanidinoacetate | H₂NC(═NBz)NHCH₂COOCH₃ |
| G16 | N-phenylacetyl ethyl guanidinoacetate | H₂NC(═NPc)NHCH₂COOC₂H₅ |
| G17 | N-phenylacetyl methyl guanidinoacetate | H₂NC(═NPc)NHCH₂COOCH₃ |
| G18 | N-(2-acetoxybenzoyl) methyl | H₂NC(═NAb)NHCH₂COOCH₃ |
| | guanidinoacetate | |
| G19 | N-(2-acetoxybenzoyl) ethyl | H₂NC(═NAb)NHCH₂COOC₂H₅ |
| | guanidinoacetate | |
| C20 | N-isobutanoyl ethyl guanidinoacetate | H₂NC(═NisoBa)NHCH₂COOC₂H₅ |
| G21 | N-isobutanoyl methyl guanidinoacetate | H₂NC(═NisoBa)NHCH₂COOCH₃ |
| G22 | N-acetyl methyl guanidinoacetate | H₂NC(═NAc)NHCH₂COOCH₃ |
| G23 | N-(pyroGlutamyl) methyl | H₂NC(═NPg)NHCH₂COOCH₃ |
| | guanidinoacetate | |
| G24 | N-(pyroGlutamyl) ethyl guanidinoacetate | H₂NC(═NPg)NHCH₂COOC₂H₅ |

In one embodiment, the composition is administered topically. In another embodiment, the composition is administered systemically, or by superficially subcutaneous injection.

In particular embodiments, the pain, itch, or eczema is associated with a medical condition, disorder, disease including arthritis (e.g., osteoarthritis, psoriatic arthritis, etc.), dental pain, itch, eczema, lipoma, muscle pain, itch, eczema, pharyngitis, sprain, trauma, sunburn, or thermal burns.

### DETAILED DESCRIPTION OF THE PRESENT DISCLOSURE

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the present disclosure. Such discussion is not an admission that any or all of these matters form part of the prior art with respect to any inventions disclosed or claimed herein.

Unless defined otherwise, all technical and scientific terms used herein have the same meaning as commonly understood by one of ordinary skill in the art to which the present disclosure pertains, published patent applications and publications cited herein are incorporated by reference as if set forth fully herein.

It must be noted that as used herein and in the appended claims, the singular forms “a,” “an,” and “the” include plural reference unless the context clearly dictates otherwise.

Throughout this specification and the claims that follow, unless the context requires otherwise, the word “comprises” and “comprising”, will be understood to imply the inclusion of a stated integer or step or group of integers or steps, but not the exclusion of any other integer or step or group of integer or step. When used herein, the term “containing” or “including” or sometimes when used herein with the terms “having”.

When used here, “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising”, containing “, including”, and “having”, whenever used herein in the context of an aspect or embodiment of the present disclosure can be replaced with the term “consisting of” or “consisting essentially of” to vary scopes of the disclosure.

As used herein, the term “treatment” or “treating” refers to amelioration, improvement, prophylaxis, or reversal of a disease or disorder, or at least one discernible symptom hereof. In certain embodiment, “treatment” or “treating” refers to amelioration, improvement, prophylaxis, or reversal of at least one measurable physical parameter related to the disease or disorder being treated, not necessarily discernible in or by the mammal or subject. In another embodiment, “treatment” or “treating” refers to inhibiting or slowing the progression of a disease or disorder, either physically, e.g., stabilization of a discernible symptom, physiologically, e.g., stabilization of a physical parameter, or both. In yet another embodiment, “treatment” or “treating” refers to delaying the onset of a disease or disorder.

In some embodiments, compounds and composition as described in the present disclosure are administered as a preventative measure. As used herein, the “prevention” or “preventing” refers to a reduction of the risk of acquiring a given disease or disorder.

Common or certain knowledge, scientific and medical terminologies can be readily found via internet, textbooks of chemistry, biochemistry, medicinal chemistry, pharmacology, dermatology and general medicine. The following are some examples: Robert K. Murray et al. eds. “Harper's Illustrated Biochemistry” 26th edn. Vol. I-II McGraw Hill, 2003. Laurence L. Brunton et al eds. “Goodman & Gilman's The Pharmacological Basis of Therapeutics” 12th edn. McGraw Hill Medical, New York, 2011. Anthony S. Fauci et al. eds. “Harrison's Principles of Internal Medicine” 17th edn. McGraw Hill Medical, New York, 2008. Abba J. Kastin, Ed “Handbook of Biologically Active Peptides” CRC Press 2009.

Certain abbreviations and terms used herein include: Ac (acetyl), Ba (butanoyl), isoBa (isobutanoyl), Bo (benzyloxycarbonyl), Bz (benzoyl), Fo (formyl), Hd (hexadecanoyl, He (hexanoyl), Hp (heptanoyl), Le (linoleic), Na (nonanoyl), Oa (octanoyl), Pa (propanoyl), Pc (phenylacetyl), Pe (pentanoyl), Pg (pyroglutamyl); ethyl ester, OEt; propyl ester, OPr; and methyl ester, OMe; NH(C═NH)NHNH₂; aminoguanidine radical; —NHNH(C—NH)NH₂; aminoguanidine radical with different attachment.

In one general aspect, the present disclosure relates to a guanidinoacetic acid or its derivative of Formula (2),

H₂NC(═NR₁)NHCH₂COR₂ FORMULA (2)

with or without the combination of other pharmacological agents, and a composition comprising the same, or optionally, a pharmaceutically acceptable salt thereof; wherein,
R₁ is an acyl radical having up to 29 carbon atoms;
R₂ is OR₃, NHR₄, or any amino group containing a radical having up to 10 carbon atoms;
R₃ is H, an alkyl, aralkyl, or aryl radical having up to 19 carbon atoms; and
R₄ is H, OH, an alkyl, aralkyl, aryl or acyl radical having up to 19 carbon atoms.

A typical R₂ can be OH, OEt, NHOH, NH₂, NHNH₂, N═NHNHAc, NHCONH₂, NH(C═NH)NH₂, NH(C═NH)NHNH₂, NHNH(C═NH)NH₂, NH(C═NH)NHAc, NHNH(C═NH)NHAc or (H₃C)₂N(C═N)N(CH₃)₂.

A typical acyl radical and abbreviation suitable for use in the present disclosure includes, but is not limited to, Ac (acetyl), isoBa (isobutanoyl), Bo (benzyloxycarbonyl), Bz (benzoyl), Fo (formyl), Hd (hexadecanoyl), Hp (heptanoyl), Le (linoleic), Ln (linolenic), Na (nonanoyl), Oa (octanoyl) Pa (propanoyl), Pc (phenylacetyl), Pe (pentanoyl), and Pg (pyroglutamyl).

In another general aspect, the present disclosure relates to use of the guanidinoacetic acid or its derivative of Formula (2),

H₂NC(═NR₁)NHCH₂COR₂ FORMULA (2)

with or without the combination of other pharmacological agents having synergetic or synergistic effect, or the use of the pharmaceutical composition comprising the same, or optionally, the pharmaceutically acceptable salt thereof.

In some embodiments, the present disclosure relates to a method for treating pain, itch, eczema, or discomfort associated with an inflammatory cosmetic or a medical condition, disorder, or disease, the method comprising administering to a human subject in need thereof a composition comprising a guanidinoacetic acid or its derivative of Formula (2),

H₂NC(═NR₁)NHCH₂COR₂ FORMULA (2)

with or without the combination of other pharmacological agents, and a composition comprising the same, or optionally, a pharmaceutically acceptable salt thereof; wherein,
R₁ is an acyl radical having up to 29 carbon atoms;
R₂ is OR₃, NHR₄, or any amino group containing a radical having up to 10 carbon atoms;
R₃ is H, an alkyl, aralkyl, or aryl radical having up to 19 carbon atoms; and
R₄ is H, OH, an alkyl, aralkyl, aryl or acyl radical having up to 19 carbon atoms.

In some embodiments, the composition is administered to the human subject in a therapeutically effective amount.

In some embodiments, the composition is administered to the human subject in a cosmetically effective amount.

In some embodiments, the guanidinoacetic acid or its derivative of Formula (2) is selected from the representative compounds in Table 1:

TABLE 1
Representative Compounds

| Number | Name | Formula |
|---|---|---|
| G10 | guanidinoacetic acid | H₂NC(═NH)NHCH₂COOH |
| G11 | N-acetyl ethyl guanidinoacetate | H₂NC(═NAc)NHCH₂COOC₂H₅ |
| G12 | N-propanoyl ethyl guanidinoacetate | H₂NC(═NPa)NHCH₂COOC₂H₅ |
| G13 | N-propanoyl methyl guanidinoacetate | H₂NC(═NPa)NHCH₂COOCH₃ |
| G14 | N-benzoyl ethyl guanidinoacetate | H₂NC(═NBz)NHCH₂COOC₂H₅ |
| G15 | N-benzoyl methyl guanidinoacetate | H₂NC(═NBz)NHCH₂COOCH₃ |
| G16 | N-phenylacetyl ethyl guanidinoacetate | H₂NC(═NPc)NHCH₂COOC₂H₅ |
| G17 | N-phenylacetyl methyl guanidinoacetate | H₂NC(═NPc)NHCH₂COOCH₃ |
| G18 | N-(2-acetoxybenzoyl) methyl | H₂NC(═NAb)NHCH₂COOCH₃ |
| | guanidinoacetate | |
| G19 | N-(2-acetoxybenzoyl) ethyl | H₂NC(═NAb)NHCH₂COOC₂H₅ |
| | guanidinoacetate | |
| C20 | N-isobutanoyl ethyl guanidinoacetate | H₂NC(═NisoBa)NHCH₂COOC₂H₅ |
| G21 | N-isobutanoyl methyl guanidinoacetate | H₂NC(═NisoBa)NHCH₂COOCH₃ |
| G22 | N-acetyl methyl guanidinoacetate | H₂NC(═NAc)NHCH₂COOCH₃ |
| G23 | N-(pyroGlutamyl) methyl | H₂NC(═NPg)NHCH₂COOCH₃ |
| | guanidinoacetate | |
| G24 | N-(pyroGlutamyl) ethyl guanidinoacetate | H₂NC(═NPg)NHCH₂COOC₂H₅ |

In some embodiments, the compound of Formula (2) is not guanidinoacetic acid.

In some embodiments, the compound of Formula (2) is a guanidinoacetic acid derivative.

In certain embodiments, the guanidinoacetic acid derivative is of Formula (2), wherein R₁ is an acyl radical selected from the group consisting of acetyl, propanoyl, isobutanoyl, benzoyl, phenylacetyl, 2-acetoxybenzoyl, and pyroglutamyl; R₂ is OR₃; and R₃ is methyl or ethyl.

In certain embodiments, the guanidinoacetic acid derivative of Formula (2) is G11, G12, G13, g14, G15, G16, G17, G18, G19, G20, G21, G22, G23, or G24.

In certain embodiments, the composition comprises G11, which is N-acetyl ethyl guanidinoacetate.

In certain embodiments, the composition comprises G22, which is N-acetyl methyl guanidinoacetate.

In certain embodiments, the composition comprises G19, which is N-(2-acetoxtbenzoyl) ethyl guanidinoacetate.

In certain embodiments, the composition comprises G16, which is N-phenylacetyl ethyl guanidinoacetate.

In some embodiments, the composition comprises at least 1% by weight or volume, based on a total weight or volume of the composition, of the guanidinoacetic acid derivative of Formula (2).

In certain embodiments, the composition comprises about 1% to about 10%, such as about 1%, 2%, 3%, 4%, 5%, 6%, 7%, 8%, 9%, 10%, or any number in between thereof, by weight or volume, based on a total weight or volume of the composition, of the guanidinoacetic acid derivative of Formula (2).

In some embodiments, the composition is topically administered.

In some embodiments, the composition is systemically administered, such as subcutaneous injection, parenteral injections, oral or nasal spray, and under the tongue administration.

In some embodiments, the method provides anti-pain effect.

In some embodiments, the method provides treatment for itch or eczema.

In certain embodiments, the eczema is selected from the group consisting of atopic eczema and inflammatory eczema.

In certain embodiments, for preventive measure before the sign of eczema shows up in the human subject in need thereof, the method comprises administering to the human subject a composition comprising a guanidinoacetic acid derivative selected from the group consisting of N-acetyl ethyl guanidinoacetate and N-acetyl methyl guanidinoacetate, and without being combined with an anesthetic.

In some embodiments, the method further comprises administering another pharmacological agent to provide synergetic or synergistic effect.

In certain embodiments, the pharmacological agent is selected from the group consisting of acetaminophen, 2-acetoxybenzoic acid; benzophenone; betamethasone dipropionate; butoconazole; caffeic acid; caffeine; clobetasol propionate; clotrimazole; dapsone; erythromycin; gluconic acid; gluconolactone; glucuronic acid; glucuronolactone; glycolic acid; hydrocortisone; hydrocortisone 21-acetate; hydrocortisone 17-butyrate; hydrocortisone 17-valerate; hydrogen peroxide; hydroquinone; kojic acid; lactic acid; mandelic acid; minoxidil; retinal; 13-cis-retinoic acid; retinoic acid; retinol; retinyl acetate; retinyl palmitate; and triamcinolone acetonide

In one another general aspect, the present disclosure relates to guanidinoacetic acid or its derivative thereof,

H₂NC(═NR₁)NHCH₂COR₂ FORMULA (2),

or a pharmaceutically acceptable salt thereof, or a solvate thereof, wherein,
R₁ is H or an acyl radical having up to 29 carbon atoms;
R₂ is H, OR₃, NHR₄, or any other amino group containing a radical having up to 10 carbon atoms;
R₃ is H, an alkyl, aralkyl, or aryl radical having up to 19 carbon atoms;
and R₄ is H, OH, an alkyl, aralkyl, aryl, or acyl radical, where the alkyl, aralkyl, aryl, or acyl radical has up to 19 carbon atoms.

A typical acyl radical and abbreviation suitable for use in the present disclosure includes, but is not limited to, Ac (acetyl), Ba (butanoyl), isoBa (isobutanoyl), Bo (benzyloxycarbonyl), Bz (benzoyl), Fo (formyl), Hd (hexadecanoyl), Hp (heptanoyl), Le (linoleic), Ln (linolenic), Na (nonanoyl), Oa (octanoyl) Pa (propanoyl), Pc (phenylacetyl), Pe (pentanoyl), and Pg (pyroglutamyl).

In some embodiments, R₁ is H, Ac (acetyl), isoBa (isobutanoyl), Bo (benzyloxycarbonyl), Bz (benzoyl), Fo (formyl), Hd (hexadecanoyl), He (hexanoyl), Hp (heptanoyl), Le (linoleic), Ln (linolenic), Na (nananoyl), Oa (octanoyl), Pa (propanoyl), Pc (phenylacetyl), Pe (pentanoyl), or Pg (pyroglutamyl).

In some embodiments, R₁ is H, Ac (acetyl), Bz (benzoyl), or Pc (phenylacetyl).

In some embodiments, R₂ is H, OR₃, or NHR₄, preferably H, OH, OEt, OC₃H₇, NHOH, or NH₂.

In some embodiments, R₂ is NHNHR₅; and R₅ is H, OH, an alkyl, aralkyl, aryl or acyl radical, wherein the alkyl, aralkyl, aryl, or acyl radical has up to 19 carbon atoms.

In some embodiments, R₂ is NHNH₂, NHNHAc, NHCONH₂, NH(C═NH)NH₂, NH(C—NH)NHNH₂, NHNH(C—NH)NH₂, NH(C—NH)NHNHAc, NHNH(C—NH)NHAc, or (H₃C)₂N (C═N)N(CH₃)₂.

In certain embodiments, R2 is NH(C═NH)NH2, NH(C═NH)NH₂, or NHNH (C—NH) NH₂.

Representative guanidinoacetic acid or the derivatives in Formula (2) are listed in Table 1.

The phrase “pharmaceutically acceptable salt”, as used herein, means those salts of a compound of interest that are safe and effective for use in mammals and that possess the desired biological activity. Pharmaceutically acceptable salts include salts of acidic or basic groups present in the specified compounds. Pharmaceutically acceptable acid addition salts include, but are not limited to, hydrochloride, hydrobromide, hydroiodide, nitrate, sulfate, bisulfate, phosphate, acid, phosphate, isonicotinate, carbonate, bicarbonate, acetate, lactate, salicylate, citrate, tartrate, propionate, butyrate, pyruvate, oxalate, malonate, pantothenate, bituarate, ascorbate, succinate, maleate, gentisinate, fumarate, gluconate, glucaronate, saccharate, formate, benzoate, glutamate, methansulfonate, ethanesulfonate, benzenesulfonate, p-tolunesulfonate and pamoate (i.e., 1, 1′-methylene-bis(2-hydroxy-3-naphthoate) salts. Certain compounds used in the application can form pharmaceutically acceptable salts with various amino acids. Suitable base salts include, but are not limited to aluminum, calcium, lithium, magnesium, potassium, sodium, zinc, bismuth, and diethanolamine salts. For a review on pharmaceutically acceptable salts see Berge et al., 66-J. Pharm. Sci. 1-19 (1977), incorporated herein by reference.

Compounds of the present disclosure can exist in solvated and unsolvated forms. The term “solvate”, as used herein, means a physical association, e.g., by hydrogen bonding, of a compound of the application with one or more solvent molecules. The solvent molecules in the solvate can be present in a regular arrangement and/or a non-ordered arrangement. The solvate can comprise either a stoichiometric or non stichiometric amount of the solvent molecules. “Solvate” encompasses both solution-phase and isolable solvates. Compounds of the application can form solvates with water (i.e., hydrates) or common organic solvents. Exemplary solvates include, but are not limited to, hydrates, ethanolates, methanolates, and isopropanolates. Methods of solvation are generally known in the art.

Compounds of the present disclosure can be made or synthesized by any method known to those skilled in the art in view of the present disclosure. Methods of making guanidinoacetic acid derivatives, such as chemical synthesis, are well known to those of ordinary skill in the art in view of the present disclosure.

In another general aspect, the present disclosure relates to a composition comprising guanidinoacetic acid or a derivative thereof of Formula (2):

H₂NH(═NR₁)NHCH₂COR₂ FORMULA (2),

or a pharmaceutically acceptable salt thereof, and optionally a pharmaceutically or cosmetically acceptable carrier, wherein,
R₁ is H or an acyl radical having up to 29 carbon atoms;
R₂ is H, OR₃, or NHR₄, or any other amino group containing a radical having up to 10 carbon atoms;
R₃ is H, an alkyl, aralkyl or aryl radical, wherein the alkyl, aralkyl, or aryl radical has up to 19 carbon atoms; and
R₄ is H, OH, an alkyl, aralkyl, aryl, or acyl radical, wherein the alkyl, aralkyl, aryl or acyl radical has up to 19 carbon atoms.

A composition according to the present disclosure can comprise guanidinoacetic acid or any derivative thereof of Formula (2) or any pharmaceutically acceptable salt thereof or any solvate thereof described herein.

In some embodiments, R₁ is H, Ac (acetyl), Ba (butanoyl), isoBa (isobutanoyl), Bo (benzyloxycarbonyl), Bz (benzoyl), Fo (formyl), Hd (hexadecanoyl), He (hexanoyl), Hp (heptanoyl), Le (linoleic), Ln (linolenic), Na (nananoyl), Oa (octanoyl), Pa (propanoyl), Pc (phenylacetyl), Pe (pentanoyl), or Pg (pyroglutamyl).

In some embodiments, R₁ is H, Ac (acetyl), Bz (benzoyl), or Pc (phenylacetyl).

In some embodiments, R₂ is H, OR₃, or NHR₄, preferably H, OH, OMe, OEt, OC₃H₇, NHOH, or NH₂.

In some embodiments, R₂ is NHNHR₅; and R₅ is H, OH, an alkyl, aralkyl, aryl or acyl radical, wherein the alkyl, aralkyl, aryl, or acyl radical has up to 19 carbon atoms.

In some embodiments, R₂ is NHNH₂, NHNHAc, NHCONH₂, NH(C═NH)NH₂, NH(C—NH)NHNH₂, NHNH(C—NH)NH₂, NH(C—NH)NHNHAc, NHNH(C—NH)NHAc, or (H₃C)₂N(C═N)N(CH₃)₂.

In certain embodiments, R₂ is NH(C—NH)NH₂, NH(C═NH)NH₂, or NHNH (C—NH)NH₂.

Representative guanidinoacetic acid or the derivatives of Formula (2) are listed in Table 1 described above.

In some embodiments, the compound of Formula (2) is not guanidinoacetic acid.

In some embodiments, the compound of Formula (2) is a guanidinoacetic acid derivative.

In certain embodiments, the guanidinoacetic acid derivative is of Formula (2), wherein R₁ is an acyl radical selected from the group consisting of acetyl, propanoyl, isobutanoyl, benzoyl, phenylacetyl, 2-acetoxybenzoyl, and pyroglutamyl; R₂ is OR₃; and R₃ is methyl or ethyl.

In certain embodiments, the guanidinoacetic acid derivative of Formula (2) is G11, G12, G13, g14, G15, G16, G17, G18, G19, G20, G21, G22, G23, or G24.

In certain embodiments, the composition comprises G11, which is N-acetyl ethyl guanidinoacetate.

In certain embodiments, the composition comprises G22, which is N-acetyl methyl guanidinoacetate.

In certain embodiments, the composition comprises G19, which is N-(2-acetoxtbenzoyl) ethyl guanidinoacetate.

In certain embodiments, the composition comprises G16, which is N-phenylacetyl ethyl guanidinoacetate.

In some embodiments, the composition comprises at least 1% by weight or volume, based on a total weight or volume of the composition, of the guanidinoacetic acid derivative of Formula (2).

In certain embodiments, the composition comprises about 1% to about 10%, such as about 1%, 2%, 3%, 4%, 5%, 6%, 7%, 8%, 9%, 10%, or any number in between thereof, by weight or volume, based on a total weight or volume of the composition, of the guanidinoacetic acid derivative of Formula (2).

According to embodiments of the present disclosure, the composition comprises a cosmetically or therapeutically effective amount of guanidinoacetic acid or a derivative thereof of Formula (2) or a pharmaceutically acceptable salt thereof or a solvate thereof. In view of the present disclosure, standard procedure can be performed to evaluate the effect of administration of a composition to a subject (e.g., determine whether a clinically observable beneficial effect is achieved), thus allowing a skilled artisan to determine the therapeutically effective amount of guanidinoacetic acid, or the derivative thereof, of the pharmaceutically acceptable salt thereof, or the solvate thereof. A clinically observable beneficial effect can be a situation that, when a composition of the present disclosure is administered to a subject prior to the symptoms which are to be treated become observable, the symptoms are prevented from occurring, or subsequently occur to a lesser degree than without administration of the composition.

As used herein, the term “carrier” refers to any excipient, diluent, buffer, stabilizer, or other material well known in the art for pharmaceutical or cosmetical formulations. The carriers in particular are non-toxic and should not interfere with the efficacy of the active ingredient. The carriers include excipients and/or additives suitable for use in the pharmaceutical or cosmetical compositions known in the art, e.g., as listed in “Remington: The Science & Practice of Pharmacy”, 19th ed., Williams & Williams, (1995), and in the “Physician's Desk Reference”, 52nd ed., Medical Economics, Montvale, N.J. (1998), the disclosures of which are entirely incorporated herein by reference. Any conventional carrier or excipient may be used in the compositions of the present disclosure.

The choice of a particular carrier or excipient, or combinations of carriers or excipients, will depend on the mode of administration being used to treat a particular patient or type of medical condition or disease state. In this regard, the preparation of a suitable composition for a particular mode of administration is well within the scope of those skilled in the pharmaceutical or cosmetical arts. Additionally, the carriers or excipients used in the compositions described herein may be commercially-available. By way of further illustration, conventional formulation techniques are described in Remington: The Science and Practice of Pharmacy, 20th Edition, Lippincott Williams & White, Baltimore, Maryland (2000); and H.C. Ansel et al., Pharmaceutical Dosage Forms and Drug Delivery Systems, 7th Edition, Lippincott Williams & White, Baltimore, Maryland (1999).

All formulations of the pharmaceutical or cosmetical composition disclosed herein can be produced by the conventional methods in the pharmaceutical or cosmetical field. For example, the active ingredient can be mixed with one or more excipients, then to make the desired formulation.

### EXAMPLES

The following examples illustrate the compositions and methods of the present disclosure. The examples do not limit the invention, but are provided to teach how to make useful controlled release drug delivery compositions.

### Example 1

### Formulation Vehicles

“WEP 442” represents water 40 parts, ethanol 40 parts and propylene glycol 20 parts by volume; “WEP 433” represents water 40 parts, ethanol 30 parts and propylene glycol 30 parts by volume; “WEP 244” represents water 20 parts, ethanol 40 parts and propylene glycol 40 parts by volume; “EP 55” represents anhydrous composition with ethanol 50 parts and propylene glycol 50 parts by volume; and “WP 82” represents water 80 parts and propylene glycol 20 parts by volume.

G11 N-acetyl ethyl guanidinoacetate 3 g was dissolved in a 97 ml solution made of water 40 ml, ethanol 40 ml and propylene glycol 20 ml. The composition thus prepared contains 3% N-acetyl ethyl guanidinoacetate in WEP solution G11X3WEP442.

Similarly, G22X3WEP442 was formulated by dissolving 3 grams N-acetyl methyl guanidinoacetate in 97 ml solution made of water 40 ml, ethanol 40 ml and propylene glycol 20 ml.

For the cream composition G11X3H, N-acetyl ethyl guanidinoacetate 3 grams was dissolved in water 20 ml, propylene glycol 20 ml, and the solution thus obtained was mixed with 57 grams of hydrophylic ointment.

Similarly, the cream composition, G22X3H is formulated by dissolving N-acetyl methyl guanidinoacetate 3 g in 20 ml of water and 20 ml of propylene glycol, and the solution thus obtained was mixed with 57 grams of hydrophylic ointment. The composition thus formulated contained 3% N-acetyl methyl guanidinoacetate.

### Example 2

### Evaluation of Analgesic and Anti-Itch Effectiveness

Five scales were used to evaluate the efficacy of analgesic or anti-itch effect: 0 (zero) for no effect; 1+ (25%), which represents partial relief of pain, itch, eczema for less than 6 hours; 2+ (50%), which represents substantial but incomplete relief of pain, itch, eczema for less than 6 hours; 3+ (75%), which represents complete relief of pain, itch, eczema for less than 6 hours; and 4+ (90-100%), which represents complete relief or eradication of pain, itch, eczema for more than 6 hours.

### Example 3

### Effect on Cervicle Stenosis

A male human subject, age 90, had suffered severe pain in his left side neck almost once a week. The pain felt deep and tight to the neck area, and with much difficulty of turning the neck. His MRI of cervical spine showed aging related spinal stenosis at C4-5 and C5-6, which indicates spinal canal becomes too narrow for the nerve fibers.

Usually, the pain episode happened when the subject was sitting for too long doing computer work. A few drops of solution G11 X 3 WEP442 was topically applied to the neck area without occlusion. The pain usually disappeared within a few minutes and did not show up again until the next day.

At different day, when the neck started to tighten up again with deep painful feeling, the male subject this time topically applied a few drops of the solution G22x3 WEP442 to the neck area, again the pain disappeared within a few minutes and did not return until the next day.

The above results show that guanidinoacetic acid derivative can be used to treat cervical pain.

### Example 4

### Effect on Tooth Ache

A male human subject, age 90, had deep and painful tooth ache at tooth number 26 one night. He was desperately looking for something to relieve the pain. A solution of G11 X 3 WEP442 was prepared as described in the above examples, and applied topically with a dropper to the gum surrounding tooth number 26. After two consecutive applications, the deep pain disappeared completely, and subject was able to sleep. The pain still hadn't returned when he visited the dentist several days later where the tooth with cavity was removed.

The above results show that guanidinoacetic acid derivative can be used to treat tooth aches.

### Example 5

### Effect on Itch/Eczema

A male human subject, age 90, developed intense itching and rash on upper thighs of both legs after eating too much seafood pizza. No further seafood was consumed after the rash appeared, but the rash and itch intensified for the next few days and the rash became small vesicles, which are typical lesions of eczema. Hot water appeared to stop the itch for 12 hours, but had no effect on the rash. Hydrocortisone 1% cream did not stop the itch, nor reduced or eliminated the rash. The male subject topically applied G22x3H cream to the affected area. A few minutes after application, the itch stopped completely for more than 6 hours. With continued use of G22x3H cream, the vesicles and eczema disappeared completely, and the skin on the thighs returned to normal after one week.

The above results show that guanidinoacetic acid derivative can be used successfully for itch and eczema.

### Example 6

### Hydrophilic Cream Made of Shea Butter

Unlike petrolatum, shea butter can be formulated as a non-greasy cream which can incorporate with various ingredients for topical use. For example, a mixture of shea butter 30 g, stearyl alcohol 3g, PEG-40 stearate 5 g, glyceryl monostearate 5 g, water 30 ml and propylene glycol 10 g were heated to 80° C., and the resulting mixture were stirred until a uniform white cream was obtained.

Various topical agents can be incorporated into a shea butter cream. For example, N-acetyl guaonidino ethyl acetate 3% cream can be readily formulated by dissolving G11, in water 20 ml and propylene glycol 10 ml, and the solution thus obtained was mixed with 70 g of shea butter cream until a creamy product, G11x3Hs, was obtained. Similarly, G22x3Hs was readily formulated.

### Example 7

### Effect on Lower Back Pain

A male human subject, age 52, had moderate to serve lower back pain due to strenuous exertion. After the subject applied G19 X 3 WEP442 topical application on the affected lower back without occlusion, the pain was reduced by 80% allowing the subject to function normally. The results lasted for 24 hours.

The above result shows that guanidinoacetic acid derivative can be used to treat lower back pain.

### Example 8

### Effect on Foot Pain

A male human subject, age 53, had severe foot pain from straining the foot from extensive walking. After he applied G16 X 3 WEP442 topical application on the affected foot pain area, the pain was reduced by 100%. The results lasted for 24 hours.

The above result shows that guanidinoacetic acid derivative can be used to treat foot pain.

### Example 9

### Effect on Joint Pain

A male human subject, age 53, had right knee moderate joint pain due to strenuous exertion and history of knee issues from dislocation and periodic joint pain. After he applied G19 X 3 WEP442 topical application on the affected knee pain without occlusion, the pain was reduced by 100% allowing the subject to function normally for 24 hours.

The above result shows that guanidinoacetic acid derivative can be used to treat joint pain.

### Example 10

### Effect on Shoulder Pain

A male human subject, age 55, has ongoing minor to moderate periodic shoulder discomfort from previous injured joints. After he applied G19 X 3 WEP442 topical application on the shoulder discomfort without occlusion, the pain was reduced by 100% allowing the subject to function normally for 4 hours. Minor discomfort returned and the reapplied G19 X 3 WEP442 topical application as necessary.

The above results show that guanidinoacetic acid derivative can be used to treat shoulder pain.

### Example 11

### Effect on Itch

A female human subject, age 54, had an itchy spot on her neck. After she topically applied G19 x 5H cream, there was complete relief from itch within 15 minutes. The result lasted for 24 hours. The above result shows that guanidinoacetic acid derivative can be used to treat itch.

### Example 12

### Effect on Sciatic Nerve Pain

A male human subject, age 52, has been suffering from severe pain in his left leg. The pain felt deep at the hip area, causing much difficulty sleeping during the night. This pain was diagnosed as sciatic nerve pain. A few drops of solution G19 X 5 WEP442 was topically applied to the hip area without occlusion. The pain and inflammation were reduced within 10-15 minutes, which was not happening with prescription strength Voltarin (Diclofenac Sodium) cream. The pain did not go away completely, but G19 X 5 WEP442 helped to reduce the pain and inflammation.

We claim:

1. A method for treating pain, itch or eczema in a human subject in need thereof, the method comprising administering to the human subject a composition comprising a guanidinoacetic acid derivative of Formula (2):
H₂NC(═NR₁)NHCH₂COR₂ FORMULA (2)
wherein, R₁ is an acyl radical selected from the group consisting of acetyl, propanoyl, isobutanoyl, benzoyl, phenylacetyl, 2-acetoxybenzoyl, and pyroglutamyl; R₂ is OR₃; and R₃ is methyl or ethyl.

2. The method of claim 1, wherein the guanidinoacetic acid derivative is selected from the group consisting of:
G11 N-acetyl ethyl guanidinoacetate
H₂NC(═NAc) NHCH₂COOC₂H₅
G12 N-propanoyl ethyl guanidinoacetate
H₂NC(═NPa) NHCH₂COOC₂H₅
G13 N-propanoyl methyl guanidinoacetate
H₂NC(═NPa)NHCH₂COOCH₃
G14 N-benzoyl ethyl guanidinoacetate
H₂NC(═NBz)NHCH₂COOC₂H₅
G15 N-benzoyl methyl guanidinoacetate
H₂NC(═NBz)NHCH₂COOCH₃
G16 N-phenylacetyl ethyl guanidinoacetate
H₂NC(═NPc)NHCH₂COOC₂H₅
G17 N-phenylacetyl methyl guanidinoacetate
H₂NC(═NPc)NHCH₂COOCH₃
G18 N-(2-acetoxybenzoyl) methyl guanidinoacetate
H₂NC(═NAb)NHCH₂COOCH₃
G19 N-(2-acetoxtbenzoyl) ethyl guanidinoacetate
H₂NC(═Ab)NHCH₂COOC₂H₅
C20 N-isobutanoyl ethyl guanidinoacetate
H₂NC(═NisoBa)NHCH₂COOC₂H₅
G21 N-isobutanoyl methyl guanidinoacetate
H₂NC(═NisoBa)NHCH₂COOCH₃
G22 N-acetyl methyl guanidinoacetate
H₂NC(═NAc)NHCH₂COOCH₃
G23 N-(pyroGlutamyl) methyl guanidinoacetate
H₂NC(═NPg)NHCH₂COOCH₃
G24 N-(pyroGlutamyl) ethyl guanidinoacetate
H₂NC(═NPg NHCH₂COOC₂H₅.

3. The method of claim 1, wherein the composition comprises N-acetyl ethyl guanidino acetate.

4. The method of claim 1, wherein the composition comprises N-acetyl methyl guanidino acetate.

5. The method of claim 1, wherein the composition comprises N-(2-acetoxtbenzoyl) ethyl guanidinoacetate.

6. The method of claim 1, wherein the composition comprises N-phenylacetyl ethyl guanidinoacetate.

7. The method of claim 1, wherein the composition comprises at least 1% by weight or volume, based on a total weight or volume of the composition, of the guanidinoacetic acid derivative of Formula (2).

8. The method of claim 1, wherein the composition comprises about 1% to about 10% by weight or volume, based on a total weight or volume of the composition, of the guanidinoacetic acid derivative thereof of Formula (2).

9. The method of claim 8, wherein the composition comprises about 3% by weight or volume, based on a total weight or volume of the composition, of the guanidinoacetic acid derivative thereof of Formula (2).

10. The method of claim 8, wherein the composition comprises about 5% by weight or volume, based on a total weight or volume of the composition, of the guanidinoacetic acid derivative thereof of Formula (2).

11. The method of claim 1, wherein the composition is topically administered.

12. The method of claim 1, wherein the composition is systemically administered.

13. The method of claim 12, wherein the composition is administered by subcutaneous injection.

14. The method of claim 1, wherein the method provides anti-pain effect.

15. The method of claim 1, wherein the method provides treatment for itch or eczema.

16. The method of claim 1, wherein the method further comprises administering another pharmacological agent to provide synergetic or synergistic effect, and the pharmacological agent is selected from the group consisting of acetaminophen, 2-acetoxybenzoic acid; benzophenone;
betamethasone dipropionate; butoconazole; caffeic acid; caffeine; clobetasol propionate;
clotrimazole; dapsone; erythromycin; gluconic acid; gluconolactone; glucuronic acid;
glucuronolactone; glycolic acid; hydrocortisone; hydrocortisone 21-acetate; hydrocortisone 17-butyrate; hydrocortisone 17-valerate; hydrogen peroxide; hydroquinone; kojic acid; lactic acid;
mandelic acid; minoxidil; retinal; 13-cis-retinoic acid; retinoic acid; retinol; retinyl acetate;
retinyl palmitate; and triamcinolone acetonide.

17. The method of claim 1, wherein the eczema is selected from the group consisting of atopic eczema and inflammatory eczema.

18. The method of claim 15, which is for preventive measure before the sign of eczema shows up in a human subject in need thereof, wherein the method comprises administering to the human subject a composition comprising a guanidinoacetic acid derivative selected from the group consisting of N-acetyl ethyl guanidinoacetate and N-acetyl methyl guanidinoacetate, and without being combined with an anesthetic.
